(19) United States
(12) Patent Application Publication
(10) Pub. No.: US 2015/0250707 A1
(43) Pub. Date: Sep. 10, 2015

(54) Composition and method for hair care products and bleaching

(71) Applicant: Sabrina Lee, Oak Park, MI (US)

(72) Inventor: Sabrina Lee, Oak Park, MI (US)

(21) Appl. No.: 13/999,484

(22) Filed: Mar. 4, 2014

(51) Int. Cl. A61K 8/97; A61Q 5/08

(52) U.S. Cl. CPC A61K 8/97; A61Q 5/08; A61K 2800/10

(57) ABSTRACT

A composition and method for hair bleaching and hair care products. Any hair type, texture or previous chemical application can successfully achieve levels as high as 10 with the use of double 40 volume developer on hair as fragile as relaxed African hair in the shortest time frame of 2-3 weeks with no breakage or damage short term or long term effects. Most caucasian hair can accomplish and be sustain high levels because of hair structure and natural higher base colors. But long term no one is exempt from laws of nature a highly alkaline leveled chemical resulting in thinning fine shredded shaft and ends, think Dolly Parton without the wigs if you've ever seen her, its not pretty. The method is a “step by step” process physically applying the ingredients to hair and scalp and to bleach of emollients, proteins and surfactants of low molecular weight, hydrolyzed, cold pressed and temperature controlled. The Method and Composition allow the hair balance during the long term effects. Preferable ingredients, rich in fatty acids, antioxidants, uv protection, hydration complexes, regeneration, rejuvenating enzymes, absorption and filmogenics, a combination that I prefer that is original.

### RELATED APPLICATIONS

This nonprovisional patent application claims the benefit of provisional patent application U.S. Ser. No. 61/851,720, filed Mar. 13, 2013, which is hereby incorporated by reference in its entirety.

### BACKGROUND OF THE INVENTION

1. Field of the Invention

The present invention relates to the hair care and bleaching of the hair, a composition and method that protects and prevents long term and short term damage of the chemical and natural processes of the hair on hair as fragile as African relaxed hair. Tones may be accomplished in a 1 day service, for deeper tones in the shortest time frame of 2-3 weeks accomplishing level 10 using double 40 as an option of developer for heavier red pigments level 5 or less.

2. Description of the Related Art

Various compositions and methods exist for bleaching preparations. 2002 “L'Oreal”, 1995 “The One”, used various silicone emulsions prepped by washing the hair. A disadvantage of this previous method for preparations using water as an aid to lift and deposit disrupts the p.h. of the hair before the application of bleach which is level 10 in p.h. With my invention the p.h. is controlled and preserved with preferable surfactants, antioxidants and hydrating complexes. Application done before application of bleach.

Various methods exist that do not require you to shampoo the pretreatment as well. The use of keratinous fibers, silicones, and organic oils.

The disadvantage of the current methods using keratin fibers and organic oils used today are the combinations. Fibers are rough in texture and adds more stress to the hair during an already stressful period. Organic oils are typically used alone which is not preferable heavy in consistency. Some have the combination of silicone and oil, but silicone is widely known for its damaging effects as well as its over weight properties, finer hair even through shampooing can be weighed down.

Various intense protein treatment mask and an intense moisture treatment mask are all applied to hair for maintenance. The masks are recommended after chemical processing to insure balance. The daily set are low in molecular weight as well to ensure richness without the heavy and overbearing consistency. The formulations are systematically combined, 90% moisturizers or ingredients with moisturizing characteristics and 10% surfactants or ingredients with surfactant characteristics. The proteins intense treatment mask is 30% protein or ingredients of keratinization characteristics, 60% emollients or ingredients with moisturizing characteristics and 10% surfactants or ingredients with surfactant characteristics. The disadvantages are within the formulations, the consistency of emollients, proteins and surfactants, the combination and the target of the p.h. are preferable. The knowing when to apply these ingredients even as early as the beginning of the alkaline application through methods that are step by step preferably indentifying its most intense time of need.

### SUMMARY OF THE INVENTION

In accordance with the invention, the foregoing advantages have been achieved through the present combinations and methods that make it possible for any hair texture, type or previously chemically applied hair to successfully achieve tones with the use of bleach with volumes as high as double 40 developer with no disruptions or breakage during the chemical and natural processes. In the shortest amount of time of 2-3 weeks for levels 5 and lower that vary in depth and warmth opposed to the current method of over highlighting the hair over the course of 5-8 months.

In accordance with another aspect of the invention, the foregoing advantages have also been achieved through a “step by step” preferable methods that create a supreme nourishing barrier before, after and during the chemical application and combinations of preferable surfactants, emollients and proteins that have low molecular weight and proteins that are nonresistant with hydrating properties. Combinations preferably used that target the needs of the p.h. of the hair to control the chemical cycle and growth processes and effects. Maintaining its proper balance to result in the hairs health longevity.

In accordance with another aspect of this invention, preferable ingredients are applied physically to the hair, a barrier, before application of bleach. The preferable ingredients are low molecular weight rich emollients, fatty acids that are locked into a hydrating surfactant. Providing protection to hair and scalp.

In accordance with another aspect of this invention an additive is physically applied to the bleach. Made with the preferable combination of surfactants, emollients, proteins that result to a reconstructing, fatty acid, antioxidant, rejuvenating hydrating surfactants. This creates nourishing elements within the bleach intermixed and distributed within the oxidation process of the bleach. Measurements of additive per scoop of bleach without the developer.

In accordance with another step to the method of the invention a moisture neutralizing shampoo is applied after rinse out of bleach. Apply protein neutralizer to hair let sit for 5 minutes. Apply toner then rinse thoroughly. Apply moisturizing neutralizing shampoo after the toner is rinsed allow 5 minutes to sit and rinse. These are preferable methods. An intense moisturizing mask or an intense protein mask of preferable ingredients is applied to hair after shampooing methods are complete. Upon the individuals hair needs they are applied and used for maintenance from there on.

### DETAILED DESCRIPTION OF THE INVENTION

The embodiment of the procedure, recognized as an original combinated method, consists of ingredients applied to hair before, during and after. Physically ingredients are applied to hair for optimum protection, choices of ranges in surfactants, filmogenics, preferably a coating classification as a barrier. Ingredients physically applied to bleach, ingredients kept fresh preferably mixing ratio 8 grams per oz not including the volumizer. Apply bleach properly, rinse followed by an application of neutralizing shampoo qualities preferably to sit 5-10 mins. Rinse then apply conditioner treatment that preferably consist of plant based protein and reconstructors only to the newly colored hair. Rinse then apply the barrier again before application of toner. Apply toner, rinse as usual then preferably apply the neutralizing ingredient again let sit 5 mins then begin to apply either Select Techniques Bleaching Systems moisturizing mask or Select Techniques Bleaching Systems protein mask or the combination of both to preferably target the preferred target of ph control, chemical cycles and growth cycles.

The embodiment of the hair care formulation of the present invention includes emollients and the species of safflower seed oil (carthamus tinctorius), macadamia ternifolia seed oil, safflower oleosomes (HLB 6), Flax seed gel (linum usitatissimum) 2 tbs whole seeds, ½ cup of water 48 celsius, 24 hour, gelatin result. Olive oil, argan oil (argania spinosa), avocado oil (persea americana) cold pressed 25 celsius. Jojoba oil (shuub simmondsia chinesis), grape seed extract (vitis vinifera grapes), coconut oil (cocos nucifera) cold pressed 25 celcius. Hydrolyzed collagen (gelatin) raw materials the gelatin purchased at, “Zhenghou Sigma Chemical Co., Ltd. No. 8 Sanquan Rd. Jinshui Distr. 450000 China Henan.” Aloe Barbadensis leaf juice, collected from a cold press of 30 Celsius and shea oil (butyrospermum parkii seed oil) collected during the buttering process.

The preferable proteins of the formulation include hydrolyzed wheat protein 0.5% concentration refrigerated at 25 Celsius molecular weight at 1000, hydrolyzed corn oil (zea mays) unsaponifiable, hydrolyzed oat protein, aqua hydrolyzed vegetable protein-pg-propyl silanetriol, raw material of aqua hydrolyzed vegetable . . . “Stradco Corporation 27 Hiram Road Richmond Hill, Ontario 24c 9E6, Canada.” Quinoa hydrolyzed (keen wa) 1% in concentration cold pressed, raw material purchased at, “Tri-K Industries' Akulu Marchon Pty Ld. Chemserve Place Turnberry Place Office Park 48 Grosvner Rd. Bryanston, 2021 South Africa.

The preferable sealants are lanolin, raw material of lanolin purchased at, “Shanghai Sungo Technology and trade Co., Ltd. Room 320, F/3, Tonghua Mansion, No. 345 Jinxaing Rd. Jinqiao Export processing zone, Podong, Shanghai, 201206, China 20/206.” Petroleum and jojoba oil.

The intense moisturizing mask treatment preferable ingredients are safflower seed oil 10%, safflower oleosomes 7%, flax seed gel 5%, olive oil 10%, argan oil 12%, avocado oil 10%, jojoba oil 5%, coconut oil 15%, grape seed extract 5%, hydrolyzed collagen 1%, aloe vera leaf juice 10% and shea oil 5% and macadamia ternifolia 5%, silk amino protein 5%. The preferable ingredients of the moisturizer treatment provide an intense combination of low molecular weight emollients that serve purposes of the p.h. controlling factors of the moisturizing needs and demands.

The protein treatment mask preferable ingredients are hydrolyzed wheat protein 5%, hydrolyzed corn oil 5%, hydrolyzed oat protein 5%, hydrolyzed veg-protein-pg-propyl silanetriol 3%, silk amino acids 15%, Quinoa hydrolyzed 1%, safflower seed oil 5%, safflower oleosomes 10%, macadamia ternifolia seed oil 7%, flax seed gel 6%, olive oil 4%, argan oil 5%, avocado oil 5%, jojoba oil 4%, grape seed oil 5%, coconut oil 5%, hydrolyzed collagen 1%, aloe vera leaf juice 5%, shea oil 4%. Again creating a protein based moisturizer, preferable ingredients that target the needs of the p.h. during times of higher deficiencies. A system of preferable proteins that behave like emollients in retaining moisture, the hairs highest demand. Systematically combined to not over bear each other but to work together in common factors and points preferably.

I claim:

1. Claim method and composition of a bleaching technology that gives the ultimate hair care to coloring procedures including bleach with multiple chemical applications, no short term or long term damage, wherein said a 9 step by step procedure preferably used for optimum protection in the bleaching procedure preferably targeting the growth cycles and the chemical cycles for all hair types, previous chemical application optimized hair health and integrity proper balance between emollients, protein and surfactants, plant based, preferably to not limit the option of double 40 developer to achieve tones as light as 10 in the shortest amount of time of 2-3 weeks [if necessary the length of time may be in shorter time as well] to provide no breakage or disruption on hair, even as fragile as relaxed African hair

2. The method and composition of claim 1 where in physical application a 9 step by step procedure of a protecting barrier preferably of surfactant filmogenic qualities and plant based ingredients is applied to hair and scalp for optimum protection that consists of targeting the deteration process of the alkaline at the preferable initiation of the process by using a method there of

3. The method and composition of claim 1 where in the 9 step by step procedure includes preferably steps originally combinated together to work as a proper function of ultimate control of damage at any phase are as follows
[a] apply a plant based filomogenic surfactant to scalp and hair
[b] apply a plant based oil preferably to bleach 8 oz per scoop of bleach without the application of bleach
[c] apply bleach properly, process time then rinse
[d] apply neutralizer shampoo allow sitting time 5 mins, rinse
[e] apply preferably a plant based organic reconstructor and protein based conditioning treatment only to newly colored hair allow processing time 5 mins, rinse
[f] apply barrier again onto hair from scalp to ends, apply toner, rinse
[g] then proceed with Select Techniques intense moisturizing mask

4. The composition and method of claim 2 where in said an intense moisturizing mask is applied to hair after coloring application and is used for at home maintenance that gives the ultimate resolution to the hairs constant loss of moisture through environment, styling, and chemical, the preferable combination of emollient and surfactants work together as one system regenerating and rejuvenating moisture while locked into place

5. The composition and method of claim 2 where in said an intense moisturizing mask classifications antioxidants, emollients, surfactants, protein, rejuvenents, regeneratives, preferable ratios for moisturizing mask 90% moisturizers or protein of moisturizing characteristics and 10% surfactants or surfactant characteristics, applied to hair to meet the hairs demand for constant moisture these preferable ingredients are high in concentration and immediately replenish and replace its need, each moisturizer penetrates and stabilizes each layer of the hair shaft preferably to fill in each deficiency of moisture a preferable variety that restores the hair protecting it from its 70% limitation

6. The composition and method of claim 2 the intense moisturizing mask treatment preferable ingredients are safflower seed oil 10%, safflower oleosomes 7%, flax seed gel 10%, olive oil 10%, argan oil 12%, avocado oil 10%, jojoba oil 0.2%, coconut oil 15%, grape seed extract 5%, hydrolyzed collagen 1%, aloe vera leaf juice 10% and shea oil 5%, 5% silk amino proteins, the preferable ingredients of the moisturizer treatment provide an intense combination of low molecular weight emollients that serve purposes of the p.h. controlling factors of the moisturizing needs and demands replenishing hair immediately with highly concentrated emollients that lock in the moisture as well, ingredients such as flax seed gel and jojoba oil are more cuticle layer ingredients while aloe vera and shea oil are more cortex layer ingredients, my preferable combination covers cuticle, cortex and medulla

7. the composition and method of claim 3 where in said a protein mask treatment is used on hair after coloring procedure and at home for maintenance to replenish and maintains hairs proper keratination levels, during the aggressive phase of chemical deteration and slower phase of growth cycles, my combination of proteins are preferably non resistant, has moisturizing qualities due to the high demand of moisture and are preferably the most agreeable keratin with our own natural keratin giving a truer similarity of strength and flexibility, these preferable proteins stay longer in the hairs shaft and with the added surfactants they are locked

8. the composition and method of claim 3 where in said the protein mask consists of preferable ingredients of 30% protein or keratinizing characteristics 60% emollients or moisturizing characteristics, 10% surfactants or surfactant characteristics, the protein is cut by 50% with a moisturizer because the proteins are in such high concentration the moisturizer balances the preferable proteins by easing the keratins attachment and locks the keratins attachment into place surrounded by moisturizing agents and immediately supplies the proteins with moisturizing characteristics immediately so that the keratin is not looking for the environment to supply which causes an oxidizing processes on its own, cuticles are now open for the hairs demand, preferably

9. The method and composition of claim 3 The protein treatment mask preferable ingredients are hydrolyzed wheat protein 5%, hydrolyzed corn oil 5%, hydrolyzed oat protein 5%, hydrolyzed veg-protein-pg-propyl silanetriol 3%, silk amino acids 15%, Quinoa hydrolyzed 1%, safflower seed oil 5%, safflower oleosomes 10%, macadamia ternifolia seed oil 7%, flax seed gel 6%, olive oil 4%, argan oil 5%, avocado oil 5%, jojoba oil 4%, grape seed oil 5%, coconut oil 5%, hydrolyzed collagen 1%, aloe vera leaf juice 5%, shea oil 4% again creating a protein based moisturizer, preferable ingredients that target the needs of the p.h. during times of higher deficiencies a system of preferable proteins that behave like emollients in retaining moisture, the hairs highest demand

10. The method and composition of claim 3 the protein mask preferable systematically combined to not over bear each other but to work together in common factors and points preferably, again working in the preferable 3 layer system with preferable combinated proteins that last longer on each layer sealed in with preferable surfactants proteins such as hydrolyzed wheat protein and hydrolyzed oat protein is more cortex medulla layered ingredient and hydrolyzed oat is cortex layered ingredient
